(19) United States
(12) Patent Application Publication
(10) Pub. No.: US 2006/0105402 A1
(43) Pub. Date: May 18, 2006

(54) Blood type method system and device

(76) Inventors: Gennady Rott, Ashdod (IL); Fred Samuels, Rehovot (IL); Falk Fish, Tel Aviv (IL); Ariela Ben-Zvi, Holon (IL)

Correspondence Address: Martin Moynihan;PRTSI, Inc., P.O. Box 16446, Arlington, VA, 22215, US

(21) Appl. No.: 11/272,971

(22) Filed: Nov. 15, 2005

Related U.S. Application Data
(60) Provisional application No. 60/627,162, filed on Nov. 15, 2004.

(51) Int. Cl. G01N 33/567; C12Q 1/56; C12M 1/34

(52) U.S. Cl. 435/7.21; 435/13; 435/287.2

(57) ABSTRACT

A method for the detection and/or visualization of particle agglutination in a particle suspension is provided, comprising placing a volume of the particle suspension and a volume of a solution or suspension containing an agglutinating agent at substantially the same selected location on a surface of a filter constructed so as to permit passage of individual unagglutinated particles in a direction perpendicular to the surface. A wash solution at optionally placed at substantially the same location as that of said agglutinating agent, and the surface is observed for the presence of particles. There is also provided a method for detection of agglutination reactions, such as used in blood grouping, reverse grouping and cross-matching. A device and a kit based on the invention is also claimed, which facilitates blood grouping, reverse grouping and matching in non-laboratory environment without the need for laboratory instruments.

### RELATED APPLICATIONS

This application claims priority from U.S. Provisional Patent Application No. 60/627,162, filed on Nov. 15, 2004, the contents of which are incorporated herein by reference.

### FIELD OF THE INVENTION

The present invention relates to detection of receptor-ligand interactions in general, and more particularly, to the detection of blood group antigens and their antibodies for the purpose of forward and reverse blood typing and matching as employed in transfusion medicine.

### BACKGROUND OF THE INVENTION

Particle agglutination is a widely adopted immunological method for the detection and visualization of antigen-antibody interaction due to its simplicity, rapidity and relative sensitivity (Riochet 1993). Cells in general, and red blood cells in particular, are particles which are amenable to a variety of agglutination methods. As such, agglutination of red blood cells (hemagglutination) is employed for the detection of antigens on their surface and antibodies to such antigens in blood group typing or matching as significant components in pre-blood transfusion testing procedures (Rouger 1993; Brecher 2002). The aim of pre-transfusion testing of blood is to prevent adverse reactions stemming from hemagglutination or hemolysis of blood cells due to immunological incompatibility between the blood cells of the donor and those of the recipient. “Blood Grouping” or “Blood Matching” is the series of tests employed to detect the antigenic makeup of the donor red blood cells and to predict the reaction of the recipient against such antigens.

The most clinically significant antigen system is the ABO red blood cell antigen system, which is unique inasmuch as the majority of human individuals produce antibodies to those antigens without having been actively immunized against them. Thus, an individual's red cells may display either A, B or both A and B antigens. About 40% of the population does not carry either of these antigens andis therefore, typed or grouped as “O” or Zero. The plasma or serum in blood of group O individuals has antibodies against both A and B group antigens. The plasma or serum in blood of group AB individuals, however, does not demonstrate antibodies to either A or B group antigens. Accordingly, the plasma or serum in blood of group A individuals has antibodies against B group antigens, while the plasma or serum in blood of group B individuals has antibodies against A group antigens.

Incompatibility in the ABO antigen system will result in strong adverse reactions, which can be prevented by matching the donor to the recipient. Ideally, the donor and recipient should belong to the same blood group; however, in the absence of an identical donor, an alternative blood group may be suitable as long as the recipient serum or plasma does not carry natural antibodies against the donor red cells. Thus O group is the universal donor, since its red cells will not react with either anti-A or anti-B antibodies, which are present in the blood of all other groups. Individuals of group AB can receive blood from all blood groups, since they do not have antibodies to any of them.

The Rh or “D” antigen is also tested together as part of the above “Blood Grouping”. The red cells of an individual can either carry the Rh antigen (Rh⁺ or “Rh-positive”) or not carry it (Rh⁻ or “Rh-negative”). Unlike the ABO antigen system, anti-Rh (anti-D) antibodies are not normally present in the blood Rh-negative individuals. Such antibodies nevertheless develop in Rh-negative individuals following an immunological potentiation resulting from a transfusion with Rh-positive blood or from pregnancy with an Rh-positive fetus. Modern transfusion medicine dictates matching of Rh between donor and recipient, in addition to ABO matching, in order to prevent generation of Rh antibodies in Rh-negative individuals and to prevent adverse reactions in individuals who carry anti-Rh antibodies.

Besides the A, B and Rh antigens, red blood cells may carry a variety of other antigens (see: Brecher, 2002, for a detailed discussion), which are sometimes referred to as: “sub-groups”. Similarly to the Rh antigen (and to most antigens in nature), antibodies to those antigens are not normally present in human blood, but may arise due to previous blood infusions or pregnancy with an antigen-carrying fetus. Such antibodies are referred to as “unexpected” or “rare”. Although adverse reactions due to such antigens and antibodies are rare or minor, testing for unexpected antibodies in blood recipients, and for the related antigens on donor red cells, is practiced in industrialized countries, when the supply of donor blood is sufficient and adequate.

It is, therefore, clear from the above that blood agglutination procedures play a major role in transfusion medicine and each blood donation unit and each potential recipient of blood donation undergoes various levels of tests, which may include all or part of the following:

Blood Grouping: all donated blood units and the blood of newly admitted patients and/or potential blood recipients are tested for the presence of ABO/Rh antigens on the erythrocytes.

Direct or forward grouping/typing: determination of the ABO/Rh blood type by testing the blood specimen for the presence of ABO/Rh antigens on its red blood cells with sera against the particular antigens.

Reverse grouping/typing: determination of the blood type by testing the serum or plasma fractions of the blood specimen for the presence of antibodies to the A and B antigens by using red blood cells of a defined blood type.

Cross-matching: this is the last phase of pre-transfusion testing, in which the recipient serum/plasma is reacted with the red cells of the selected donor blood unit(s). In some countries the cross-matching is not actually carried out in the laboratory but is rather done by matching computer stored testing data. In other countries, such as France, the cross-matching is carried out at the recipient's bed-side by nursing personnel.

The above tests can be executed by a variety of standard manual and automated particle agglutination methods. All particle agglutination methods involve mixing the particles with the agglutinating agent, which may be serum/plasma, or an artificially generated antibody reagent, incubating the mixture for various lengths of time and, finally, observing the mixture for the presence of agglutinated particles. In all methods, the red cells are diluted 1:10 or more as compared to their concentration in blood, and washing of the cells in order to remove plasma or serum traces is strongly recommended or outright required.

Detection of agglutination can be realized by naked eye observation of the mixture, which is preferably spread on a flat surface (Riochet, 1993). Alternatively, various means and instruments are available for enhancing the visual difference between agglutinated and non-agglutinated particles.

The “Slide Agglutination” methods can be carried out almost anywhere, without the need for any instrument. A drop of diluted red cells is mixed with a drop of serum/plasma/antibodies on a surface of a microscope slide or any other water impervious surface. The two components are mixed by a rod or by swirling the slide for a few minutes and observed carefully for agglutination. Some of the drawbacks of this approach include: (a) the requirement for a few minutes of mixing; (b) subjective visual determination of the results; (c) false positive reactions induced by drying; and (d) slides cannot be stored.

In the “Tube Agglutination” method a defined volume of the diluted blood cell suspension is mixed with a defined volume of serum/plasma/antibodies in a round bottom or V-shaped bottom transparent test tube. In the case of a negative reaction, the red cells will slowly descend into the center of the bottom of the tube and will form a clear, red “button”. In the case of a positive reaction, the agglutinated red cells form a lattice and spread all over the surface of the tube's bottom without forming a clear button. Instead a “lawn” of cells is apparent. Instead of waiting for the slow settling of the cells, current practices (Brecher, 2002; Gamma Biologicals, 2001) dictate centrifugation of the mixture of the red cells with the antibody solution followed by re-suspension. Cell clumps are clearly visible in case of a positive agglutination reaction. Tube Agglutination can also be performed in wells of micro-titration plates, thus reducing the volume of reagents and blood and increasing throughput. Some of the drawbacks of tube agglutination methods include: (a) requirement for laboratory instruments, personnel and environment; (b) subjective visual determination of the results; (c) incorrect centrifugation speed or time may result in false positive or false negative results, because cells clumps may be assumed to be immune agglutinates; (d) no direct record of the results; (e) bulky test tubes; and (f) danger of breakage and spills.

In the “Gel Filtration” method, the mix of red blood cells and serum/plasma/antibody is applied to a column of gel separation media (which can be particulate). The mixture is forced into the gel by centrifugation. Agglutinated red cells will not be able to penetrate the gel and will stay on top of the gel. Non-agglutinated cells will permeate the gel column and get to its bottom. Small size agglutinates may enter the gel column but will not get to its bottom.

One such method, described in U.S. Pat. No. 5,338,689, employs a column of gel particles and has evolved into a commercially successful product line for blood typing, available from DiaMed AG, Switzerland. The advantages of this method are: (a) relative ease of use; (b) clear interpretation of results; (c) ability to grade the agglutination level based on entry into the gel. The drawbacks are: (a) requirement for laboratory instruments, personnel and environment; (b) no direct record of the results; (c) bulky test hardware (even though the manufacturer refers to the product as a “card”, it is actually a series of test tubes in a holder and is by no means as flat as a “card”); and (d) complicated manufacturing (inserting gels into narrow tubes) which results in a high cost.

In some cases, the antibodies against red cell antigens are considered “incomplete” inasmuch as they are not able to agglutinate red cells directly but require the addition of an anti-globulin and/or anti-complement antibodies (sometimes referred to as: “Coombs' reagent”) to facilitate agglutination (Coombs et al., 1945; Brecher, 2002). This phenomenon is most prevalent when trying to detect antibodies against relatively weak antigens, such as those of blood subgroups and sometimes certain variants of the main blood groups. All above 3 methods are amenable to the additional step of subjecting the antibody-reacted red blood cells to the Coombs' reagent. However, this process entails a significant amount of manual manipulation. First, the mixture of red cells and the incomplete antibodies have to be washed thoroughly to remove all the unreacted immunoglobulin from the reaction mixture. Then, the washed, antibody-coated red cells are mixed with the Coombs' reagent and tested for agglutination employing one of the methods which are described above.

In immunological tests involving particles other than red blood cells, filters with defined pore size have been employed to easily separate between agglutinated and non-agglutinated particles (for example, U.S. Pat. Nos. 4,459,361 and 4,847,199). Until now, this approach has not been employed for agglutination methods with red blood cells as the agglutinated particles, especially as exemplified in blood grouping and blood matching procedures, despite some very early observations that the approach is feasible.

Based on preliminary observations of Castaneda (1950) with preserved bacterial cells, Malone and Stapleton (1951) showed that the lateral movement of red cells on a filter paper is affected by the addition of blood group antiserum, such that red cells mixed with non-related antiserum (e.g. A groups cells with anti-B) could travel laterally upon addition of saline, whereas red cells, which were mixed with the relevant antiserum (e.g. A cells with anti-A serum), were “fixed” into their location and did not move after addition of saline. A grouping method was proposed in which a drop of a high-titer anti-blood group serum is placed on a piece of filter paper (“blotting paper” in the original terminology). After the spreading of the serum, a drop of sample blood is placed at the same location and allowed to partially dry (“when the sheen has gone”). Finally two drops of saline are added. A positive reaction is signified by a smaller spot of blood than that of a negative reaction. A reversed procedure for the determination of the potency of anti-blood group antisera was also proposed, in which a drop of the serum was placed on the blotting paper followed by a drop of washed red cells. After partial drying, two drops of saline were added on top of the blood. The potency of the antiserum was in inverse relationship to the extent of the lateral spread of the blood cells.

Despite its relative simplicity, this method was not implemented in blood banking, except for the estimation of the quality of anti-blood group reagents in the laboratory of the authors (Dunsford and Bowley, 1955, 1967) and another location (Farr and Godwin 1955). The requirement for waiting for drying of the reagents before the final step of the test and the unclear differentiation between positive and negative reaction, based on the size of a red circle, are major drawbacks and may have been the reason for the continued disinterest in the method.

In a variation (Anderson 1970) of the technique, the blood and antiserum were mixed and incubated for 30 minutes. Hemagglutination was detected by inserting a strip of filter paper into the mix and following the wicking (i.e. lateral flow) of the mixing into the strip. In a positive reaction, the blood cells remain at the site of origin of flow, while the cell-free serum proceeds towards the end of the strip, whereas in a negative reaction, the blood cells move with the serum along the strip. The length of time required for the reaction and the need for two separate hardware components (mixing dish and a strip) are drawbacks which complicate the procedure and may induce errors.

Akers Biosciences Inc. (Thorofare, N.J., USA) improved Anderson's (1970) idea and developed it into a self contained device, described in U.S. Pat. Nos. 5,231,035 and 5,565,366, assigned to Akers Research. The cassette-like device is able to determine the blood group of a patient's blood by placing large drops inside each of the 2 openings in the top panel of the device (one for A and the other B blood group determination), waiting for 2 minutes and then adding a drop of saline. In the case of a negative reaction, the blood moves laterally and appears in a second opening (window). In the case of a positive reaction, the red blood cells will not move, and the second opening will stay clear (or will show the color of the saline wash). While the Akers device simplifies blood grouping to such an extent that it can be employed out of the laboratory (for example at the bedside) by non-blood-banking-professionals, it has some disadvantages: (a) it requires a relatively large volume of blood; (b) it requires the operator to time the steps; (c) it allows only for forward blood grouping and not for cross matching (visualizing activity of recipient's antibodies against donor's red blood cells); (d) the appearance of the results may be misleading: a positive result is denoted with absence of red color in the results window, whereas a negative reaction ends with the appearance of a red color; and (e) results have to be visualized exactly 1 minute after the addition of the saline wash. This requirement precludes storing the reacted device as a stable record.

It is clear that the standard blood grouping and cross-matching methods are quite labor intensive, error prone and require professional personnel to operate them in a laboratory environment. Since there is a need to conduct blood grouping procedure outside the laboratory by non-blood-banking-professional operators (such as a nurse in a bedside cross-matching in France), products were developed aiming to simplify the procedure and to eliminate operational errors. Although these products do indeed simplify blood testing procedure to a certain degree, they are not completely error free, and may be complicated for some personnel (Rachel and Plapp, 1990; Migeot et al., 2002).

### SUMMARY OF THE INVENTION

The background art does not teach or suggest a quick, simple, and accurate blood testing procedure involving an easily portable device, based on perpendicular movement through filters of defined pore sizes, use of which requires little or no specialized training, and which provides directly recordable results.

The present invention overcomes the deficiencies of the prior art by providing a method and device for conducting a variety of blood grouping, reverse grouping and cross-matching tests in a simple manner, which is suitable for use by non-professional personnel in a non-laboratory environment. In addition, the results can optionally be directly preserved and stored/filed for future reference.

According to one aspect of the present invention, there is provided a method for the detection and/or visualization of particle agglutination in a particle suspension, comprising placing a volume of the particle suspension and a volume of a solution or suspension containing an agglutinating agent at substantially the same selected location on a surface of a filter, the filter being constructed so as to permit passage of individual unagglutinated particles in a direction perpendicular to that of the surface; optionally placing a wash solution at substantially the same location as that of the agglutinating agent; and observing the surface at that selected location for the presence of particles.

Optionally and preferably, a volume of particle suspension is placed at the selected location prior to placing of a volume of a solution or suspension containing an agglutinating agent.

Optionally and preferably, the filter comprises a porous surface of a porous body, the pores of the surface being sized as to allow passage of at least individual particles.

Optionally and more preferably, the porous surface is inherently absorbent or comprises a porous layer attached to an absorbent material.

According to further features in the described preferred embodiments, a water soluble film is situated between the porous layer and the absorbent material.

According to further features in the preferred embodiments, the method further comprises drying the filter.

Optionally and preferably, the volume of particle suspension and the volume of solution or suspension of agglutinating agent each comprise a deliverable volume. Optionally and more preferably, the deliverable volume comprises at least a microliter.

Optionally and preferably, the particles of the particle suspension are coated with a member of a binding pair (MBP). Optionally and more preferably, the agglutinating agent comprises a MBP.

According to still further features in the described preferred embodiment, the particles comprise at least one of natural particles or synthetic particles.

According to yet further features in the preferred embodiments, the particles comprise a detectable label. Optionally and preferably, the label is selected from the group comprising pigments, radioactive materials, magnetic or paramagnetic materials, fluorophores, and luminescent materials

Optionally and preferably, the particle suspension comprises a first blood product.

According to further features in the preferred embodiments, the first blood product comprises whole blood.

According to further features in the preferred embodiments, the first blood product comprises a blood component.

Optionally and preferably, the blood component is in suspension.

According to further features in the preferred embodiments, the first blood product comprises red blood cells.

According to yet further features in the preferred embodiments, the first blood product comprises at least one selected from the group comprising unwashed red blood cells, undiluted red blood cells or unwashed and undiluted red blood cells.

According to still further features in the preferred embodiments, the method of the present invention is operative without centrifugation or pre-mixing of the first blood product with the particle suspension or the agglutinating agent.

According to further features in the preferred embodiments, the agglutinating agent comprises a second blood product.

According to further features in the preferred embodiments, the agglutinating agent comprises an antibody to a blood group.

According to yet further features in the preferred embodiments, the agglutinating agent comprises a serum or plasma.

According to further features in the preferred embodiments, the volume of solution or suspension containing an agglutinating agent is placed at the selected location prior to placing of the volume of particle suspension.

For the purpose of forward blood typing, the filter is optionally and preferably impregnated with agglutinating agent. Further optionally, the filter is impregnated with a reagent selected from the group comprising an anti-globulin reagent and an anti-complement reagent.

According to further features in the preferred embodiments, the reagent comprises Coombs reagent.

According to further features in the described preferred embodiments, the wash solution is a salt solution. Optionally and preferably, the salt solution is isotonic. Optionally and more preferably, the salt solution is saline solution. Optionally and more preferably, the saline is buffered. Optionally and more preferably, the buffered saline is phosphate buffered saline.

According to further features in embodiments of the present invention, the wash solution further comprises an additional washing component. Optionally and preferably, the additional washing component comprises a polymer. Optionally and more preferably, the polymer is selected from the group comprising Poly Ethylene Glycol and dextran sulfate sodium salt. Optionally and more preferably, the concentration range of the polymer is appropriate for maintenance of osmotic balance. Optionally and most preferably, the concentration range is from about 0.0001 to about 20% w/v.

According to further features in the described preferred embodiments, the wash solution further comprises at least one of a detergent and a surface active material. Optionally and preferably, the detergent comprises polyoxyethylene-10-tridecyl ether. Optionally and more preferably, the detergent or surface active agent has a concentration in the range of from 0.0001 to 0.1 % w/v. Optionally and most preferably, the concentration is in the range of from 0.001 to 0.01% w/v.

According to another aspect of the present invention, there is provided a device for detection and/or visualization of particle agglutination in a sample, comprising a filter constructed so as to permit passage of individual, unagglutinated particles placed on a surface of said filter, in a direction perpendicular to that of said surface.

Optionally and preferably, the device further comprises a mesh positioned on an upper surface of the filter.

According to further features in preferred embodiments of the present invention, the device further comprises an additional filter for receiving the agglutinating agent and removing particles from the agglutinating agent. Optionally and preferably, the additional filter is removed after addition of the particle-containing agglutinating agent and before the particle-containing sample is added to the first filter.

Optionally and preferably, the agglutinating agent comprises an antibody in whole blood or a blood component.

According to yet another aspect of the present invention, there is provide a kit for performing the method of the present invention, the kit comprising a filter for receiving the sample; an agglutination agent also for being placed on the filter; and optionally a washing solution for being placed on the filter after the agglutination agents and the sample.

Optionally and preferably, the kit further comprises a meter for detecting and/or measuring an agglutination reaction. Optionally and more preferably, the meter comprises a light meter which comprises a light source for transmitting light onto the porous surface of the filter, and a light sensor so positioned as to measure light reflected or scattered by the porous surface.

Optionally and preferably, the kit further comprises a converter for converting the measured light to a visual signal, and a display for displaying the signal.

The kit may optionally comprise at least one additional light sensor and/or at least one additional light source.

Optionally and preferably, the kit of the present invention further comprises a processing circuit.

Optionally and preferably, the kit of the present invention further comprises a holder for the filter.

Optionally and preferably, the light source provides colored light.

According to further features in preferred embodiments of the present invention, the kit further comprises an additional filter for receiving the agglutinating agent and removing particles from the agglutinating agent. Optionally and preferably, the additional filter is removable.

According to yet a further aspect of the present invention, there is provided a kit for performing the method of the present invention, the kit comprising a filter for receiving the sample and the agglutination agent, wherein the filter is optionally impregnated with a reagent; and optionally a washing solution for being placed on the filter after the agglutination solution and the sample.

Optionally and preferably, the reagent comprises Coombs' reagent.

Optionally and preferably, the reagent, impregnating the filter is the agglutinating agent.

According to still a further aspect of the present invention, there is provided a device for detecting a presence or absence of an agglutination reaction between a plurality of test components, the device comprising a filter, having an upper surface and constructed such that the direction of flow of the test components is perpendicular to the upper surface, the test components being applied to the upper surface, and optionally subjected to a wash procedure, wherein test components having undergone the agglutination reaction are detectable on the upper surface.

Optionally and preferably, the test components comprise at least one of whole blood or blood fractions or blood components.

According to further features in the described preferred embodiments, the device features a filter, which may optionally comprise one or more layers. The dimensions of the filter (preferably including at least one of thickness or area) are preferably designed to permit the direction of flow of the test components to be perpendicular to the surface on which these components are applied. The components may optionally be blood or blood fractions or components, but may also optionally include any mixture of a plurality of different components to be separated. Preferably, the components are reacted in some manner before or during application to the surface of the filter, such that separation occurs differently according to the outcome of the reaction. For example, for blood components, the reaction may optionally be agglutination, such that if the blood (or components thereof) agglutinates, the test components do not enter the filter (or enter only a short distance).

Optionally and more preferably, the presence or absence of the reaction is determined visually, most preferably by viewing the filter with the naked eye. Optionally, a meter may be used. Alternatively, other types of labels or reporters may be used, for example (optionally and preferably) with a meter for detecting the presence of the label or reporter. Various exemplary labels and reporters are described in greater detail below, but any suitable label or reporter may be used and could be selected by one of ordinary skill in the art. It should be noted that a meter could optionally be used with or without a label or reporter, as designed by one of ordinary skill in the art.

According to further features in the described preferred embodiments, the determination of results optionally and preferably involves no subjective factor or otherwise preferably involves little subjectivity on the part of the tester.

According to yet further features, the device may optionally be easily portable.

According to still further features, the device may optionally easily be used by non-professional operators.

According to further features, the method optionally and preferably does not require premixing of the reaction components.

According to further features, whole, unwashed blood may optionally be preferably used.

According to further features, a small volume of sample blood is optionally and preferably required.

According to further features in the described preferred embodiments, the method is optionally and preferably devoid of a centrifugation step.

According to further features, timing of the reaction stop is optionally not required.

According to further features, a direct record of results is optionally obtained.

An advantage of the present invention is that it is optionally and preferably suitable for both blood grouping and for cross-matching.

A feature of the present invention is that the direction of flow of the test components is preferably perpendicular to the surface of a porous body.

An advantage of the present invention is that the method and device are simple to use.

A further advantage of the present invention is that false positive results are avoided.

A further advantage of the present invention is that results are obtained quickly and accurately, preferably with a very low margin of error.

In the present specification, a number of terms are discussed hereinunder, for the purposes of description only and without any intention of being limiting. Unless otherwise defined, all technical and scientific terms used herein have the same meaning as commonly understood by one of ordinary skill in the art to which this invention belongs.

Binding Pairs (BP)—receptor-ligand pairs, including but not limited to antigen-antibody, complementary nucleic acids, lectin-carbohydrate pair, enzyme-substrate and others.

Member of binding pair (MBP)—a member of a Binding Pair, e.g. an antibody is an MBP of the antigen-antibody binding pair.

Blood typing—see” “blood grouping” herein.

Agglutinating agent—a material including but not limited to one which causes particles to bind to each other.

Drop—optionally and preferably, a volume of liquid heavy enough to fall as a single mass from an aperture of a dispensing device, including but not limited to a pipette, needle, bottle or any other container, tube or vessel with a thin, protruding opening, or optionally a solid object, wherein the volume of the drop is optionally and preferably, but not necessarily, from about 20 to about 50 μL in volume.

Porous Body—a mass of material which has pores in it. Non-limiting examples of a porous body include, but are not limited to, a filter, filter paper, absorbent paper, etc.

Filter—A device including but not limited to a porous material or mass, including but not limited to paper or sand, through which a liquid or gas can be passed in order to separate the fluid from at least a portion of suspended particulate matter, or any other absorptive material through which a liquid or gas can be passed for such separation; or a device containing such a porous material.

Lateral movement—movement of, for example, liquid and/or particles (optionally suspended or dispersed in the liquid), optionally and preferably on the surface of or inside a body, including but not limited to, a flat bibulous (i.e. porous or otherwise absorptive) body (including but not limited to membrane, paper) and parallel to the surface of the body.

Perpendicular movement—movement of, for example, liquid and/or particles suspended in such liquid through a body, including but not limited to a flat bibulous (i.e. porous or otherwise absorptive) body (including but not limited to a membrane or paper) and perpendicular to the surface of the body.

Flow Through—see “Perpendicular movement” above.

Porous surface—surface with pores or interstices which admit the passage of, for example, gas or liquid, but which do not allow passage of at least a portion of particles suspended or dissolved in the liquid or gas, wherein the particles either have size larger than that of the pores or adhere to the surface of the pores.

Blood grouping—determination of the ABO/Rh blood type, by testing methods including, but not limited to, testing the blood specimen for the presence of ABO/Rh antigens on its red blood cells, or by testing the serum or plasma fractions of the blood specimen for the presence of antibodies to the A and B antigens.

Blood typing—see: Blood Grouping.

Direct or forward grouping/typing—determination of the ABO/Rh blood type by testing the blood specimen for the presence of ABO/Rh antigens on its red blood cells with antibodies against the particular antigens.

Reverse grouping/typing—determination of the blood type by testing the serum or plasma fractions of the blood specimen for the presence of antibodies to the A and B antigens by using red blood cells of a defined blood type.

Cross matching—The last phase of pre-transfusion testing, in which the recipient serum/plasma is reacted with the donor's blood or its red cells.

Matching—see “cross matching” above

Pretransfusion cross matching—see “cross matching” above.

Sub groups—include, but are not limited to, antigens on red blood cells, which do not belong to the ABO/Rh antigen system and are genetically inherited independently of the ABO/Rh antigens.

Unexpected antibodies—antibodies against non ABO/Rh antigens that are found, for example, in the serum/plasma of human blood.

Label—tracer; a relatively easily detectable substance or moiety added or bound to, for example, a chemical, biological, or physical entity to facilitate its detection or visualization. Examples of labels, which are employed in the biomedical field, include, but are not limited to: pigments, radioactive isotope, magnetic or paramagnetic materials fluorophores, luminescent materials, enzymes, particles.

Similar location—preferably includes, but is not limited to a position which is substantially close to a location at which a volume of solution or suspension was previously placed, but may optionally also include an adjacent or nearby location.

### BRIEF DESCRIPTION OF THE DRAWINGS

The invention is herein described, by way of example only, with reference to the accompanying drawings:

FIG. 1 shows results of blood grouping tests, performed in accordance with the teachings of the present invention; and

FIG. 2 shows a schematic representation of a device and kit constructed according to the teachings of the present invention.

### DESCRIPTION OF THE PREFERRED EMBODIMENTS

The present invention provides a method and device for the detection or visualization of particle agglutination of a plurality of test components (agglutination reaction), for example for detecting an antigen-antibody interaction. The device preferably features a filter, having an upper surface. The direction of flow of the test components is mainly perpendicular to the upper surface. The perpendicular direction of flow is preferably maintained by limiting the surface area relative to the volume of specimen and wash reagent. As a non-limiting example, a surface area of from about 0.2 to about 0.25 cm² may optionally and preferably be employed for specimens of 50 μL or less. The test components are applied to the upper surface, such that if the test components undergo the agglutination reaction, the visible and/or otherwise detectable component of the test, which serves as the reporter (for example, the red blood cells according to preferred embodiments of the invention) does not appreciably traverse the filter in the perpendicular direction, so that it is visible and/or otherwise detectable on the surface after the optional wash procedure.

The method optionally and preferably involves placing a drop of suspension of the particles on the surface of the filter, placing a drop of a solution or suspension containing an agglutinating agent at the same or similar location, and optionally placing a wash solution at the same or similar location. The filter is selected such that the pores enable passage of single blood cells, perpendicular to the surface of the filter, but not of clumped cells following agglutination. Agglutinated cells therefore remain on the surface of the filter and are detectable by the presence of a red spot at the site of reaction. It should be noted that the agglutinating agent is preferably placed at substantially the same selected location as the test sample (test component or components), such that the agglutinating agent is able to react with the test sample. Thus the term ‘substantially the same selected location’ refers to a location which enables the agglutinating agent to react with the test sample.

Alternatively a plurality of vertically stacked filters may optionally be used, preferably with a water soluble film for controlling a rate of fluid transfer at least between a first filter and at least one filter in the stack below. The first filter receives both members of a binding pair (MBP). MBP are receptor-ligand pairs such as antigen-antibody, complementary nucleic acids, lectin-carbohydrate pair and others, as described in greater detail below. Accordingly, the agglutinating agent can be the complementary member of the MBP, such that the particle agglutination reaction and device can be used for the detection/determination of a member of the binding pair. Preferably, the first filter receives both members sequentially. If agglutination occurs, washing of the first filter with wash solution will not result in washing of the MBP perpendicularly through the first filter onto the second filter. Agglutination may optionally be detected visually or according to detection of a label.

A single sufficiently thick filter may also optionally be used, optionally with a water soluble film impregnated within the filter. The filter is selected such that lateral movement along the filter is minimized, such that at least a substantial portion of movement of the non-agglutinated particles occurs in a direction perpendicular to that of the surface of the filter, while agglutinated particles remain on the surface of the filter.

The present invention may be used for the purposes of blood grouping and cross-matching. The invention provides a method and device which can used outside the laboratory by non-professional operators, which does not require premixing of the reaction components, which uses whole, unwashed blood, which provides a clear color signal in case of a positive reaction, and which can be stored as a record.

Contrary to the prior art methods discussed above, which rely on the lateral flow of the test components over a filter, the inventors have found that if the direction of movement is through the filter (i.e. the direction is perpendicular to the surface of the filter), filters can be advantageously employed for hemagglutination methods (including blood grouping and matching) with some clear advantages over existing methods: (a) differentiation between agglutination and non-agglutination is clear: a red colored spot appears in case of a positive reaction and no spot develops in case of a negative reaction (or no reaction); (b) the red blood cells and their agglutinating agent need not be mixed and/or incubated for any period of time before placing them on the filter; (c) centrifugation is not required to separate the agglutinated red cells from the non-agglutinated ones in order to facilitate visualization of the results; (d) whole blood can be employed instead of a diluted (1 to about 5%) suspension of washed erythrocytes; and (e) the reaction filter can be dried and stored/filed as an unequivocal record of the reaction and its results. The filter used in the system of the present invention has suitable dimensions and properties to ensure that at least a significant proportion of particle movement occurs in a direction perpendicular to that of the surface of the filter.

According to a preferred embodiment of the present invention, there is provided a method for the detection/visualization of particle agglutination, comprising: placing a drop of a suspension of the particles on a porous surface of a porous body, the dimensions and pores of that surface being selected so as to allow perpendicular passage of individual particles; placing a drop of a solution or suspension containing an agglutinating agent at the same or similar location of the drop of particle suspension; optionally placing a wash solution at the same or similar location of the drops; observing the surface at this location for the presence of particles, such presence indicates that agglutination of the particles did occur. By optionally drying the porous material, such dried material can be stored as a record.

According to an alternative embodiment of the present invention, the order of the first two steps of the previous embodiment may be reversed, so that the method for blood grouping preferably comprises placing a drop of a solution containing antibodies to blood group antigens on a porous surface, the pores and dimensions of that surface so sized as to allow at least perpendicular passage of individual red blood cells; placing a drop of a suspension of red blood cells, optionally at the same or similar location of the drop of antibody solution; placing a wash solution on the surface, optionally at the same or similar location of the drops; observing the surface at the location for the presence of red color.

The wash solution is optionally and preferably an osmotic solution, more preferably an isotonic solution. More preferably, the wash solution is a saline solution, and most preferably a buffered saline solution. According to a preferred embodiment of the present invention, the wash solution comprises phosphate buffered saline.

The wash solution may optionally comprise an additional washing component or further components, including polymers, detergents or surface active agents. The additional washing component preferably is able to assist in the reduction of non-specific binding to the filter. The optional polymer is preferably selected from the group comprising polyethylene glycol and dextran sulfate sodium salt. Optionally and preferably, an optional polymer is added in a concentration range which is suitable for maintaining osmotic balance. More preferably, the concentration is in the range of from about 0.0001 to about 20% w/v. The wash solution may optionally further comprise a detergent and/or one or more surface active agents, more preferably in the concentration range of from about 0.0001 to 0.1% w/v. Most preferably, the concentration of the detergent or surface active agent is in the range of from about 0.001 to about 0.01% w/v.

The particles can optionally be coated with binding pairs (MBP). MBP are receptor-ligand pairs such as antigen-antibody, complementary nucleic acids, lectin-carbohydrate pair and others. Accordingly, the agglutinating agent can be the complementary member of the MBP, such that the particle agglutination reaction and device can be used for the detection/determination of a member of the binding pair (test components).

The particles can optionally be natural (e.g. cells or subcellular parts) or synthetic (latex, metal, metal oxide, carbon, pigment). The particles should preferably be labeled with, for example, pigments, radioactive materials, magnetic or paramagnetic materials fluorophores, luminescent materials, in order to facilitate easy detection or visualization.

In a preferred embodiment of the invention, the particles are red blood cells. In such an embodiment, the antigens in the MBP are optionally blood group antigens, such as the AB0/Rh antigen system which are located on the surface of erythrocytes and the antibodies are optionally and preferably the corresponding anti-blood group antibodies, which are present in the liquid fraction of blood.

In yet another embodiment of the invention the suspension of red blood cells, which serves as the agglutinable particles, optionally and preferably, comprises whole blood. Employing whole blood enables blood grouping to be performed by placing a drop of a suspension of red blood cells on a porous surface, the pores and dimensions of that surface selected so as to allow at least passage of individual red blood cells perpendicular to the plane of the surface; placing a drop of a solution containing antibodies to blood group antigens, optionally at the same or similar location of the drop of red blood cell suspension; placing a wash solution on the surface, optionally at the same or similar location of the drops; observing the surface at the location for the presence of red color, such color indicating that the red blood cells reacted with the antibodies and that those red blood cells carry the antigens to which the antibodies are directed, and/or that the red cells are of a blood type corresponding to the antibodies.

Hereinafter “red color” includes but is not limited to any color which has a red tone or which is reddish, or which appears to be similar to the color red, and/or which is indicative of the presence of red blood cells and/or any other blood component providing a red color, as well any color that blood can assume.

The porous surface of the porous body is optionally inherently absorbent or alternatively is a porous layer attached to an absorbent material. The porous layer and the attached absorbent material can optionally assume the shape of a flat card. The porous layer can be made from a porous membrane (e.g. cellulose, cellulose acetate or nitrate, Nuceleopore®), fabric (e.g. such as supplied by Sefar, Rüschlikon, Switzerland), mesh, bibulous materials (such as paper, obtainable for example from Whatman, Maidstone, UK), depth filtration media (such as glass fiber paper, obtainable for example from Ahlstrom, Pa., USA). Glass fiber paper is preferable, especially such paper which is reinforced with binders, such as supplied by Ahlstrom, Mount Holley Springs, Pa., USA.

In a variation of this embodiment, the porous material can be impregnated with the antibodies, dried and stored for a period of time before the blood grouping test takes place, such that a ready-for-use device is made available to be deployed instantly for grouping of a blood specimen, optionally without requiring any fresh antibody reagent. Impregnation of the porous material with the antibody can be passive, i.e. by simply adding an antibody solution to the porous material and letting it dry at an ambient or elevated temperature, and at atmospheric pressure or in a vacuum. Alternatively, impregnation may optionally involve active chemical binding of the antibodies to the matrix of the porous material by various processes well known in the art, such as (but not limited to) glutaraldehyde mediated binding to various materials, cyanogen bromide, cyanuric chloride or periodate mediated binding to polysaccharide based materials, silane mediated binding to glass. Additionally, the antibodies can optionally be first bound to particles (e.g. latex), and such antibody coated particles are then embedded in the porous material. These and additional methods are detailed by Dent and Aslam, 1998, Dean et al., 1985 and others.

The present invention provides a method for blood grouping, comprising: placing a drop of a suspension of red blood cells on a porous surface which is pre-impregnated with an antibody to a blood group; placing a wash solution at the location of the drops; observing the surface at the location for the presence of red color.

The above blood grouping embodiments also provide a method for either determination of the blood group antigens of the red cells (forward typing), or for the determination of the presence of specific anti-blood group antibodies when red blood cells of defined blood group are employed (reverse typing). In either combination, an embodiment of the invention allows the use of whole, non-separated blood as the test specimen. For the purpose of employing this embodiment for reverse typing/grouping of whole blood specimens, the red blood cells should be washed before being used in the test.

Thus, in a preferred embodiment of the present invention, there is provided a method for reverse typing/grouping of a whole blood specimen, comprising: placing a suspension of washed and defined red blood cells on a porous surface at a selected location; placing the specimen of whole blood, whose reverse type is to be determined, at the location of the defined red blood cells; further placing a wash solution at the location of the specimen of whole blood and defined red blood cells; and observing the surface at the selected location for the presence of red color.

In a further embodiment of the reverse typing application of the present invention, the following modifications to the procedure can be employed separately or in combination:

(a) Delaying the placement of the wash solution for a period of from 0.5 to 20 minutes, preferably from about 1 to about 10 minutes; and

(b) Impregnating the porous surface with diverse materials, including, but not limited to, salts, sugars, polymers and biopolymers, wherein the polymers include: PVA (Polyvinyl Alcohol), PVP (Polyvinylpolypyrrolidone), PEG (Polyethyleneglycol) at concentrations of from about 0.1 to about 10% w/v, preferably from about 0.5 to about 2% w/v.

In an alternative preferred embodiment of the blood typing application of the present invention, the invention can be employed for pre-transfusion cross-matching. Cross matching test is the final testing step before actual transfusion. In this test the medical staff checks for the presence in the recipient blood of antibodies to the donor blood.

The method of the invention can be employed to test compatibility of the recipient's plasma or serum with washed and/or diluted red blood cells from the donor's blood. However, the test procedure can be significantly simplified by employing whole, untreated blood from the donor as the source of red blood cells for the cross-matching test with the recipient's plasma or serum, so that the cross matching method optionally and preferably comprises: placing a drop of the recipient's serum or plasma on a porous surface, the pores of that surface so sized as to allow perpendicular passage of individual red blood cells; placing a drop of the donor's blood on the surface, optionally at the same or similar location of the drop of recipient's blood; placing a wash solution at the location of the drops of blood; observing the surface at the location for the presence of red color, such color indicates that agglutination of the red blood cells did occur and that the donor's blood is incompatible with the recipient's blood.

The cross matching method can also optionally be performed by reversing the order of the first two steps above. Hence, a further embodiment of the present invention provides a pre-transfusion cross-matching test for unexpected blood groups, preferably comprising placing a drop of a suspension of the donor's red blood cells on a porous surface, the pores of that surface so sized as to allow perpendicular passage of individual red blood cells; placing a drop of the recipient's serum or plasma on the surface, optionally at the same or similar location of the drop of red blood cell suspension; placing a drop of a solution containing an anti-globulin or anti-complement reagent on the surface, optionally at the same or similar location of the drop of red blood cell suspension; placing a wash solution at the location of the drops; observing the surface at the location for the presence of red color.

In order to further simplify the procedure so that it is even more suitable for bedside use, where laboratory equipment, such as a centrifuge, is not available to separate serum or plasma from the recipient's blood, the following method uses whole blood specimens from both the donor and recipient, and comprises placing a first filter, which prevents passage of blood cells out of whole blood, above a porous surface of a second filter, the pores of the second filter so sized as to allow passage of individual red blood cells; placing a drop of the recipient's whole blood on the first filter and waiting for at least part of the specimen to enter and/or be absorbed by the first filter, so that the filtrate of the whole blood wets the surface of the second filter; removing the first filter; placing a drop of the donor's blood on the surface, optionally at the same or similar location of the first filter before its removal; placing a wash solution at the location of the drop of blood; observing the surface at the location for the presence of red color, such color indicates that agglutination of the red blood cells did occur and that the donor's blood is incompatible with the recipient's blood.

In order to detect unexpected or weak blood group antigens and antibodies the typing and cross-matching procedure call for addition of an anti-globulin reagent (also called Coomb's reagent) or anti-complement reagent in order to obtain visible agglutination from such antigens and antibodies. Further embodiments of the invention facilitate simple incorporation of such reagent into the blood grouping and cross-matching methods. Thus, a blood typing method according to the invention optionally comprises: placing a drop of a suspension of red blood cells on a porous surface, the pores of that surface so sized as to allow perpendicular passage of individual red blood cells; placing a drop of a solution containing antibodies to blood group antigens on the surface, optionally at the same or similar location of the drop of red blood cell suspension; placing a drop of a solution containing an anti-globulin or anti-complement reagent on the surface, optionally at the same or similar location of the drop of red blood cell suspension; placing wash solution on the surface, optionally at the same or similar location of the drops; observing the surface at the location for the presence of red color, such color indicates that agglutination of the red blood cells did occur and that those red blood cells carry the antigens to which the antibodies are directed.

In the aforementioned methods, which include a Coomb's reagent, the order of the first three steps can be changed without derogating from the performance of the invention. In another embodiment of the invention, the Coomb's reagent can be pre-impregnated in the filter, dried and stored for a period of time before the blood grouping or cross matching test takes place, such that a ready-for-use device is made available to be deployed instantly for grouping of a blood specimen without requiring any fresh antibody reagent. According to this embodiment, the method for blood grouping or matching with a Coomb's reagent preferably comprises: placing a drop of the recipient's serum or plasma on a porous surface which is pre-impregnated with a Coomb's reagent, the pores of that surface so sized as to allow passage of individual red blood cells; placing a drop of a suspension of the donor's red blood cells on the porous surface, optionally at the same or similar location of the drop of recipient's serum or plasma; placing a wash solution on the surface, optionally at the same or similar location of the drops; observing the surface at the location for the presence of red color. Also this embodiment can optionally be fitted with a removable blood cell filter so that the recipient's whole blood can be used in the initial step.

In all above instances of the description of the method, a “drop” (of blood or serum/plasma or antibody reagent) may be substituted with a more defined volume of liquid as delivered with devices such as one of more of the following: a pipette, micropipette, capillary, dropper, dropper bottle, syringe, loop, open loop and/or any other mechanism, vessel or device for fluid handling. Those devices may be touched to the porous surface to facilitate delivery of the whole volume of liquid, which is required to carry out the test.

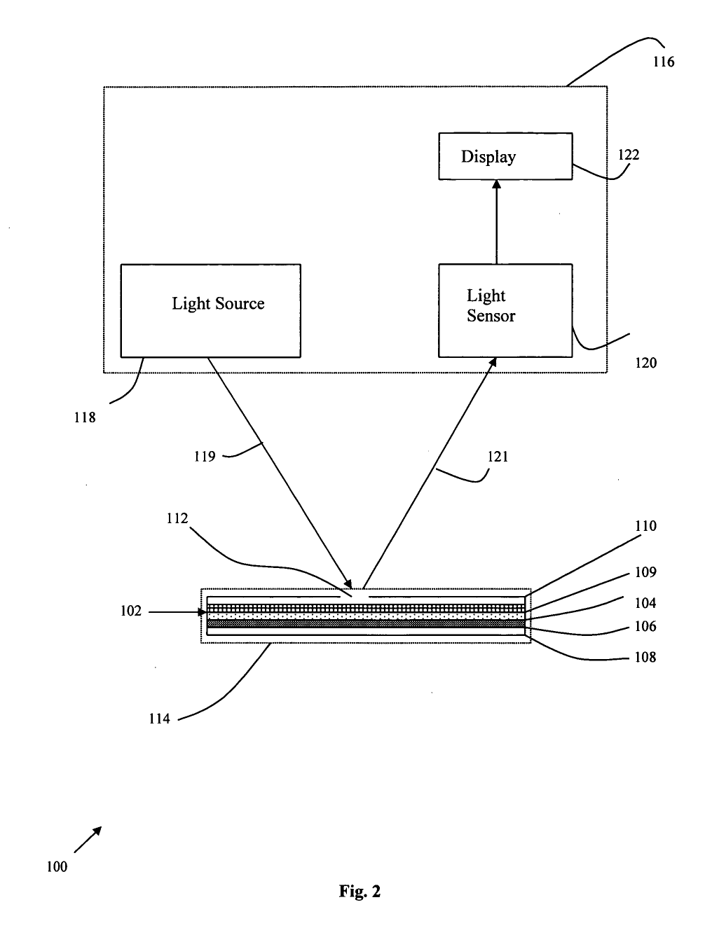

Referring to FIG. 2, a device 100 and kit may also optionally be constructed according to the teachings of the invention. The device can assume the structure of a card 102 which is formed from the filter 104 (porous layer) on top, an optional absorbent layer 106, an optional back cover 108, an optional mesh layer 109 over the filter layer 104) which facilitates rapid entry of the test components into the filter and mixing of these test components, and an optional top cover 110 with a hole 112, defining the location of placing the test specimen and conducting the test. Hole 112 in the top cover may be surrounded with wall, thus creating a well with a defined volume, to facilitate washing the reaction with defined volumes of wash solution (not shown). In addition, a water soluble film can optionally be placed between the filter and the absorbent layer to reduce the rate of liquid flow through the filter (not shown), thereby to increase sensitivity of the invention to weak antigens and/or low levels of antibody.

In a preferred embodiment of the device, the card has multiple locations for performing the tests; the locations can be optionally marked by a variety of methods, such as printing of shapes, adhesive films with cut-out shapes. The entire card can be optionally enclosed in a case 114. Device 100 may be so constructed as to allow its dismantling after use, so that the filter with the reaction zone can be removed, dried and filed/stored as a record for the results. The card may optionally have areas for writing various items of information, such as recipient and donor identification, date, device identification and more. A kit which includes one or more cards, and all, or part of the necessary reagents, wash solutions, control solutions and utensils is another embodiment of the invention.

In a preferred embodiment of the present invention, all or part of the anti-blood-groups antibodies and/or the anti-globulin and anti-complement reagents can be impregnated in the porous surface in either wet or preferably dry form. Thus, a test card with a single or multiple anti-blood group and/or anti-globulin and/or anti-complement reagents can be made available to facilitate differential identification of blood groups while preventing errors and reducing the number of manual manipulations. Such a device may optionally include positive and negative control test areas. A negative control area may optionally be accomplished by impregnating the filter at that location with a non-immune serum (such as, for example, a serum from a AB/Rh positive male, who never received any blood infusions). A positive control area may optionally be accomplished by impregnating the filter at that location with an anti-red blood cell antiserum or antibodies (such as, for example, anti-glycophorin antibodies).

In yet another embodiment of the present invention, a device with multiple test areas (such as for blood grouping of a single specimen) is provided. The device may be so constructed as to facilitate spreading of the test-specimen, and optionally of the wash solution, over all the test areas in one step. Such an embodiment can be realized by various design elements such as: (a) covering all the test areas with one piece of a mesh, preferably hydrophilic; (b) creating a capillary space above the test area by placing a non absorbent cover above all the test areas.

The device may further comprise an optional meter 116 which can interpret the results of the test without the requirement for human visual appraisal. Meter 116 preferably receives card 102 such that meter 116 is able to measure the label or reporter used to detect the agglutination reaction. Optionally, meter 116 is an optical meter and measures the amount of light, which is reflected or scattered from, or transmitted through, the location of specimen placement and determines the result accordingly. In its most basic form the meter comprises: (a) a light source 118 which directs a beam of incident light 119 onto the location of specimen placement; (b) a light sensor 120 so positioned as to measure/detect the light 121 which is reflected/scattered from, or transmitted through, the porous surface; (c) 122 a device or circuit which displays the signal derived from the light sensor.

In further embodiments of the present invention, the light source is optionally colored in order to improve detection of red blood cells. In the case in which the porous reaction surface is white, the light should optionally and preferably be non-red and more preferably blue, so that red blood cells will reduce the amount of reflected/scattered light from the white surface. Conversely, if the surface is non red, or, optionally and preferably black or blue, and the light is red, then the red blood cells will increase the amount of reflected/scattered light.

The meter may optionally have in addition one or more of the following features: display and/or printer for presenting the result(s); multiple light sensors and optionally multiple light sources for metering multiple reaction areas; a processing circuit (which may optionally include a processor and ancillary electronic devices) for analyzing the signals generated by the sensors, a holder for the filter or test card, batteries, rechargeable batteries, storage memory, time and date function, barcode reader or interface therefore, optical character recognition, communication capabilities and other features, which are well known to designers and manufacturers of medical diagnostic devices. The meter preferably further comprises a transparent or translucent window for viewing of the displayed result.

### EXAMPLES

The following examples are illustrative implementations of the methods and systems according to the present invention. These examples are not intended to be limiting in any way.

### Example 1

### Wash Solutions

Wash solutions used in generating the examples were:

A. Dulbecco's Phosphate Buffered Saline (PBS), obtained from Biological Industries, Beit Ha'emek, Israel.

B. A solution made from 1:1 diluted PBS in water with 4% w/v Poly Ethylene Glycol (PEG) 15000-20000MW (Fluka) and 0.3% w/v dextran sulfate sodium salt (Amersham Biosciences).

C. Dulbecco's Phosphate Buffered Saline (PBS) with 0.001-0.01% w/v polyoxyethylene-10-tridecyl ether (Sigma).

### Example 2

### Blood Grouping

A 0.5×0.5 cm piece of Ahlstrom #142 filter paper was placed on an absorbent pad. Two μL of whole blood were pipetted in the center of the filter and followed by 2 μL of an anti-blood group reagent (Gamma Biologicals Inc., Hosuton, Tex., USA). The filter was then washed with a few drops (from a dropper bottle) of a wash solution and dried. Alternatively, a 4 mm diameter circle of the Ahlstrom #142 filter paper, 10 μL of blood and 10 μL of the anti-blood group reagent were used followed by few drops of wash solution C from Example 1.

This test was repeated with multiple blood specimens of various blood groups (received from and pre-tested by the Central Blood Services, Israel Red Magen David, Tel Hashomer, Israel) and each of these was tested with anti-A, anti-B, anti-AB and anti-D (═Rh) blood group reagents. The A⁻ blood generated a red spot only with anti-A and anti-AB reagents. The B⁻ blood generated a spot with the anti-B and anti-AB reagents. The AB⁻ blood reacted with anti-A, anti-B and anti-AB reagent. The O⁻ blood reacted with none of the reagents and an O⁺ blood reacted only with the anti-D reagent.

### Example 3

### Blood Grouping with Dried Reagents

0.5×0.5 cm pieces of Ahlstrom #142 filter paper were placed on a non absorbent surface (bottom of empty, disposable Petri dishes). A 50 μL aliquot of an anti-blood group reagent (Gamma Biologicals Inc., Hosuton, Tex., USA) was pipetted on each of the pieces of filter paper. The Petri dishes with the anti-blood group reagents containing filter pieces were incubated overnight at 37° C. The filter pieces were dry at that time.

For testing the ability of the dried, antibody impregnated pieces to correctly identify the blood grouping of whole blood specimens, they were placed on an absorbent pad and a 1 μL aliquot of the test blood was placed in the approximate center of each of a series of filter pads. The series included pads, each impregnated with either one of anti-A, anti-B or anti-D (═Rh) reagent.

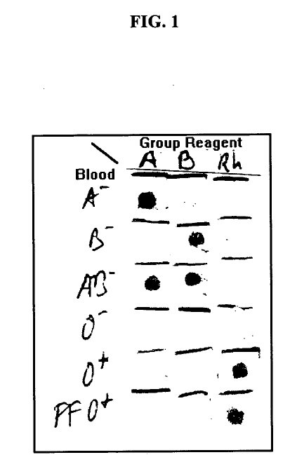

Excess blood was washed from the filter pads by placing 5-10 drops of either wash solution A or B. The filter pieces were then air dried for record keeping. The results are presented in FIG. 1. It is clear that the A⁻ blood generated a spot only on a filter piece impregnated with an anti-A reagent. The B⁻ blood generated a spot on the anti-B impregnated filter. The AB⁻ blood reacted with both anti-A and anti-B filter pieces. The O⁻ blood reacted with none of the filters and two O⁺ blood specimens reacted only with the filter pieces impregnated with an anti-D reagent.

The following modifications to the procedure appeared to result in improved signal and are optionally and preferably included in the present invention:

1. A water soluble film is placed under the filter square in order to control the rate of fluid transfer between the filter and the absorbent pad below.

2. Volume of anti-blood-group antiserum per piece of filter: 25 μL.

3. Volume of blood specimen per piece of filter: 10 μL.

4. Wash procedure: 2 drops of wash solution B (Example 1) followed by a few drops of PBS.

It should be noted that the above volumes are intended as illustrative examples only and are not meant to limit the invention in any way.

### Example 4

### Cross-Matching

Whole blood specimens, which were pre-tested for their ABO/Rh group, were obtained from the Central Blood Services, Israel Red Magen David, Tel Hashomer, Israel. Plasma fractions were derived from some of the blood specimens and served as a model for a blood recipient specimen.

0.5×0.5 cm pieces of Ahlstrom #142 filter paper were placed on an absorbent pad. 2 μL of whole donor blood specimen were pipetted in the center of the filter and followed by 10 μL of recipient plasma. The filter was then washed with a few drops (from a dropper bottle) of a wash solution and dried. A red blood dot remained on the filter after washing only when there was incompatibility between the recipient and the donor, according to the following table:

| Recipient | Donor | Result |
|---|---|---|
| Group | Group | (Red Dot) |
| A | A | − |
| A | B | + |
| A | AB | + |
| A | O | − |
| AB | A | − |
| AB | B | − |
| AB | O | − |
| O | A | + |
| O | B | + |
| O | AB | + |
| O | O | − |

Thus these results clearly indicate that the present invention has both sufficient sensitivity and also sufficient reproducibility and reliability to be useful for blood testing and/or typing.

### Example 5

### Reverse Blood Grouping

A 4 mm diameter circle of Ahlstrom #142 filter paper was placed on an absorbent pad. Fifteen μL of a 10× concentrated suspension of defined type A₁ or B test red blood cells (ID-DiaCell from DiaMed AG, s/Morat, Switzerland) were pipetted onto the center of the filter, followed by 10-50 μL of whole blood specimen. The filter was then incubated for 5 minutes at ambient room temperature, washed with a few drops (from a dropper bottle) of a wash solution A (Example 1) and observed for red color formation on the washed filter circle.

This test was repeated with multiple specimens of whole blood from various blood groups (received from and pre-tested by the Central Blood Services, Israel Red Magen David, Tel Hashomer, Israel), each of which was tested with both A₁ and B red blood cell reagents. Type A₁ blood specimens generated a red spot only with type B red cells. Type B blood specimens generated a spot only with type A₁ red cells. Type AB blood specimens did not generate a red spot with either A₁ or B red cells. Type O blood specimens generated a red spot with both A₁ or B red cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

Anderson N G (1970) “Analytical techniques for cell fractions. XVIII. Use of cellulose wicks to monitor agglutination reactions”, Anal Biochem 38:175-189
Aslam M and Dent A H (editors) (1998) “Bioconjugation: Protein Coupling Techniques for the Biomedical Sciences”, McMillan Reference LTD, London, UK
Brecher, M E (ed) (2002) “Technical Manual”, 14th Edition, American Association of Blood Banks, Bethesda, Md., USA
Castaneda M R (1950) “Surface Fixation. A new method of detecting certain immunological reactions”, Proc Soc Exptl Biol Med 73:46-49
Coombs R R A, Mourant A E and Race R R (1945) “A new test for the detection of weak and “incomplete” antibodies”, Br J Exp Path 26:255-256
Dean P D G, Johnson W S and Middle F A (Editors) (1985) “Affinity Chromatography: A Practical Approach”, IRL Press, Oxford, UK and Washington, D.C., USA
Farr A D and Godwin D C (1955) “The applications of surface fixation methods to blood group serology”, J Med Lab Tech 13:8-11
Gamma Biologicals (2001) Blood Grouping Reagents Directions for Use, Houston, Tex.
Ivor Dunsford I and Bowley C C (1955) “Techniques In Blood Grouping”, Charles C Thomas, Publisher, Springfield, Ill., USA
Ivor Dunsford I and Bowley C C (1967) ) “Techniques In Blood Grouping”, Second Edition, Volumes I and II, Charles C Thomas, Publisher, Springfield, Ill., USA
Malone R H and Stapleton R R (1951) “Blood grouping on blotting-paper”, Brit Med J I: 416 February 24
Migeot V, Ingrand I, Salmi L R, Ingrand P, (2002) “Reliability of bedside ABO testing before transfusion”, Transfusion 42:1348-55
Rachel J M, Plapp F V (1990) “Bedside blood grouping”, Med Lab Sci. 47:330-336
Riochet, D. (1993) “Particle Agglutination”, in: Masseyeff, R F, Albert, W H and Staines, N A (eds) “Methods of Immunological Analysis”, VCH Publishers, Weinheim, F D R and New York, N.Y., USA, pp. 203-214
Rouger, P. (1993) “Hemagglutination, Cell Agglutination, Mixed Cell Agglutination”, in: Masseyeff, R F, Albert, W H and Staines, N A (eds) “Methods of Immunological Analysis”, VCH Publishers, Weinheim, F D R and New York, N.Y., USA, pp. 182-202;

What is claimed is:

1. A method for the detection and/or visualization of particle agglutination in a particle suspension, comprising:
placing a volume of the particle suspension and a volume of a solution or suspension containing an agglutinating agent at substantially the same selected location on a surface of a filter, said filter being constructed so as to permit passage of individual unagglutinated particles in a direction perpendicular to that of said surface;
optionally placing a wash solution at substantially the same location as that of said agglutinating agent; and
observing said surface at said selected location for the presence of particles.

2. The method of claim 1, wherein said volume of said particle suspension is placed at said selected location prior to said placing of said volume of a solution or suspension containing an agglutinating agent.

3. The method of claim 1, wherein said filter comprises a porous surface of a porous body, the pores of said surface being sized as to allow passage of at least individual particles.

4. The method of claim 3, wherein said porous surface is inherently absorbent or comprises a porous layer attached to an absorbent material.

5. The method of claim 4, further comprising a water soluble film situated between said porous layer and said absorbent material.

6. The method of claim 1, further comprising:
drying said filter.

7. The method of claim 1, wherein said volume of said particle suspension and said volume of said solution or suspension of said agglutinating agent each comprise a deliverable volume.

8. The method of claim 7, wherein said deliverable volume comprises at least a microliter.

9. The method of claim 1, wherein the particles of said particle suspension are coated with a member of a binding pair (MBP).

10. The method of claim 9, wherein said agglutinating agent comprises a MBP.

11. The method of claim 10, wherein said particles comprise at least one of natural particles or synthetic particles.

12. The method of claim 10, wherein said particles comprise a detectable label.

13. The method of claim 12, wherein said label is selected from the group comprising pigments, radioactive materials, magnetic or paramagnetic materials, fluorophores, and luminescent materials.

14. The method of claim 1, wherein said particle suspension comprises a first blood product.

15. The method of claim 14, wherein said first blood product comprises whole blood.

16. The method of claim 14, wherein said first blood product comprises a blood component.

17. The method of claim 16, wherein said blood component is in suspension.

18. The method of claim 14, wherein said first blood product comprises red blood cells.

19. The method of claim 18, wherein said first blood product comprises at least one selected from the group comprising unwashed red blood cells, undiluted red blood cells or unwashed and undiluted red blood cells.

20. The method of claim 19, wherein the method is operative without centrifugation or pre-mixing of said first blood product with said particle suspension or said agglutinating agent.

21. The method of claim 14, wherein said agglutinating agent comprises a second blood product.

22. The method of claim 21, wherein said agglutinating agent comprises an antibody to a blood group.

23. The method of claim 21, wherein said agglutinating agent comprises a serum or plasma.

24. The method of claim 1, wherein said volume of solution or suspension containing an agglutinating agent is placed at said selected location prior to said placing of volume of said particle suspension.

25. The method of claim 24, wherein said filter is impregnated with said agglutinating agent.

26. The method of claim 24, wherein said filter is impregnated with a reagent selected from the group comprising an anti-globulin reagent and an anti-complement reagent.

27. The method of claim 26, wherein said reagent comprises Coombs reagent.

28. The method of claim 1, wherein said wash solution is a salt solution.

29. The method of claim 28, wherein said salt solution is isotonic.

30. The method of claim 29, wherein said salt solution is saline solution.

31. The method of claim 30, wherein said saline is buffered.

32. The method of claim 31, wherein said buffered saline is phosphate buffered saline.

33. The method of claim 28, wherein said wash solution further comprises an additional washing component.

34. The method of claim 33, wherein said additional component comprises a polymer.

35. The method of claim 34, wherein said polymer is selected from the group comprising Poly Ethylene Glycol and dextran sulfate sodium salt.

36. The method of claim 34, wherein concentration range of said polymer is appropriate for maintenance of osmotic balance.

37. The method of claim 36, wherein said concentration range is from about 0.0001 to about 20% w/v.

38. The method of claim 28, wherein said wash solution further comprises at least one of a detergent and a surface active material.

39. The method of claim 38, wherein said detergent comprises polyoxyethylene-10-tridecyl ether.

40. The method of claim 38, wherein said detergent or surface active agent has a concentration in the range of from 0.0001 to 0.1 % w/v.

41. The method of claim 40, wherein said concentration is in the range of from 0.001 to 0.01% w/v.

42. A device for detection and/or visualization of particle agglutination in a sample, comprising a filter constructed so as to permit passage of individual, unagglutinated particles placed on a surface of said filter, in a direction perpendicular to that of said surface.

43. The device of claim 42, further comprising a mesh positioned on an upper surface of the filter.

44. The device of claim 42, further comprising an additional filter for receiving said agglutinating agent and removing particles from said agglutinating agent.

45. The device of claim 44, wherein said agglutinating agent comprises an antibody in whole blood or a blood component.

46. The device of claim 44, wherein said additional filter is removed after addition of said particle-containing agglutinating agent and before the particle-containing sample is added to said first filter.

47. A kit for performing the method of claim 1, comprising:
a filter for receiving the sample;
an agglutination agent also for being placed on said filter; and
optionally a washing solution for being placed on said filter after said agglutination agents and the sample.

48. The kit of claim 47, further comprising a meter for detecting and/or measuring an agglutination reaction.

49. The kit of claim 48, wherein said meter comprises a light meter and said light meter comprises (a) a light source for transmitting light onto said porous surface of said filter; (b) a light sensor so positioned as to measure light reflected or scattered by said porous surface.

50. The kit of claim 49, further comprising a converter for converting said measured light to a visual signal, and a display for displaying said signal.

51. The kit of claim 49, further comprising at least one additional light sensor.

52. The kit of claim 49, further comprising at least one additional light source.

53. The kit of claim 49, further comprising a processing circuit.

54. The kit of claim 49, further comprising a holder for said filter.

55. The kit of claim 49, wherein said light source provides colored light.

56. The kit of claim 49, further comprising:
an additional filter for receiving said agglutinating agent and removing particles from said agglutinating agent.

57. The kit of claim 56, wherein said additional filter is removable.

58. A kit for performing the method of claim 1, comprising:
a filter for receiving the sample and said agglutination agent, wherein said filter is optionally impregnated with a reagent; and
optionally a washing solution for being placed on said filter after said agglutination solution and the sample.

59. The kit of claim 58, wherein said reagent comprises Coombs' reagent.

60. The kit of claim 58, wherein said reagent, impregnating the filter is the agglutinating agent.

61. A device for detecting a presence or absence of an agglutination reaction between a plurality of test components, the device comprising: a filter, having an upper surface and constructed such that the direction of flow of the test components is perpendicular to said upper surface, the test components being applied to said upper surface, and optionally subjected to a wash procedure, wherein test components having undergone the agglutination reaction are detectable on said upper surface.

62. The device of claim 61, wherein the test components comprise at least one of whole blood or blood fractions or blood components.

63. The device of claim 61, wherein the presence or absence of the agglutination reaction is detected visually.

64. The device of claim 63, wherein said presence or absence is detected by viewing the filter with the naked eye.

65. The device of claim 61, further comprising a meter for detecting the presence or absence of the agglutination reaction.

66. A method for typing of a first blood product, comprising:
placing a volume of the first blood product and a volume of a solution or suspension containing an agglutinating agent at substantially the same selected location on a surface of a filter, said filter being constructed so as to permit passage of individual unagglutinated particles in a direction perpendicular to that of said surface;
optionally placing a wash solution at substantially the same location as that of said agglutinating agent; and
observing said surface at said selected location for the presence of particles,
wherein the presence of said particles indicates the presence of an antigen-antibody interaction between said first blood product and said second blood product.

67. A method for forward typing of a first blood product of unknown antigen component, comprising:
placing a volume of the first blood product of unknown antigen component and a volume of a solution or suspension containing an agglutinating agent comprising a known antibody at substantially the same selected location on a surface of a filter, said filter being constructed so as to permit passage of individual unagglutinated particles in a direction perpendicular to that of said surface;
optionally placing a wash solution at substantially the same location as that of said agglutinating agent; and
observing said surface at said selected location for the presence of particles,
wherein the presence of said particles indicates the presence in said first blood product of an antigen to which said known antibody binds.

68. The method of claim 67, wherein said first blood product comprises red blood cells.

69. The method of claim 68, wherein said red blood cells are washed prior to placing on said surface of said filter.

70. The method of claim 68, wherein said agglutinating agent comprises a second blood product.

71. The method of claim 70, wherein said agglutinating agent comprises a serum or plasma.

72. The method of claim 70, wherein said second blood product comprises whole blood.

73. The method of claim 70, wherein said second blood product comprises a blood component.

74. The method of claim 73, wherein said second blood component is in suspension.

75. The method of claim 70, wherein said second blood product comprises red blood cells.

76. The method of claim 75, wherein said second blood product comprises at least one selected from the group comprising unwashed red blood cells, undiluted red blood cells or unwashed and undiluted red blood cells.

77. A method for reverse typing of a first blood product of unknown antibody component, comprising:
placing a volume of the first blood product of unknown antibody component and a volume of a solution or suspension containing an agglutinating agent comprising a known antigen at substantially the same selected location on a surface of a filter, said filter being constructed so as to permit passage of individual unagglutinated particles in a direction perpendicular to that of said surface;
optionally placing a wash solution at substantially the same location as that of said agglutinating agent; and
observing said surface at said selected location for the presence of particles,
wherein the presence of said particles indicates the presence in said first blood product of an antibody to which said known antigen binds.

78. The method of claim 77, wherein said first blood product comprises whole blood.

79. The method of claim 77, wherein said first blood product comprises red blood cells.

80. The method of claim 79, wherein said red blood cells are washed prior to placing on said surface of said filter.

81. The method of claim 77, wherein said agglutinating agent comprises a second blood product.

82. The method of claim 81, wherein said agglutinating agent comprises a serum or plasma.

83. The method of claim 81, wherein said second blood product comprises whole blood.

84. The method of claim 81, wherein said second blood product comprises a blood component.

85. The method of claim 84, wherein said blood component is in suspension.

86. The method of claim 81, wherein said second blood product comprises red blood cells.

87. The method of claim 81, wherein said second blood product comprises at least one selected from the group comprising unwashed red blood cells, undiluted red blood cells, unwashed and undiluted red blood cells, washed red blood cells, washed and diluted red blood cells, concentrated red blood cells or washed and undiluted red blood cells.

88. The method of claim 77, wherein said placing of said wash solution is performed at a time period of from about 0.5 to about 20 minutes after said placing of said agglutinating agent.

89. The method of claim 88, wherein said placing of said wash solution is performed at a time period of from about 1 to about 10 minutes after said placing of said agglutinating agent.

90. The method of claim 77, wherein said filter is impregnated with an agent selected from the group consisting of a salt, a sugar, a polymer, a biopolymer, and combinations and mixtures thereof.

91. The method of claim 90, wherein said polymer is selected from the group consisting of polyvinyl alcohol, polyvinylpyrrolidone and polyethyleneglycol.

92. The method of claim 91, wherein concentration of said polymer is in the range of from about 0.1 to about 10 % w/v.

93. The method of claim 92, wherein said concentration is in the range of from about 0.5 to about 2 % w/v.
